(19) United States
(12) Patent Application Publication
(10) Pub. No.: US 2013/0137701 A1
(43) Pub. Date: May 30, 2013

(54) COMBINATION

(71) Applicants: Tona M. Gilmer, Research Triangle Park, NC (US); James G. Greger, JR., King of Prussia, PA (US); Li Liu, King of Prussia, PA (US)

(72) Inventors: Tona M. Gilmer, Research Triangle Park, NC (US); James G. Greger, JR., King of Prussia, PA (US); Li Liu, King of Prussia, PA (US)

(21) Appl. No.: 13/814,799

(22) PCT Filed: Aug. 9, 2011
(86) PCT No.: PCT/US11/47019

§ 371 Date: Feb. 7, 2013

(30) Foreign Application Priority Data
Aug. 9, 2010 (US) 61371791

(51) Int. Cl. A61K 31/517; A61K 31/501

(52) U.S. Cl. CPC A61K 31/517; A61K 31/501
USPC 514/252.04

(57) ABSTRACT

The present invention relates to a method of treating cancer in a human and to pharmaceutical combinations useful in such treatment. In particular, the method relates to a cancer treatment method that includes administering N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, to a human in need thereof.

### FIELD OF THE INVENTION

The present invention relates to a method of treating cancer in a mammal and to combinations useful in such treatment. In particular, the method relates to a novel combination comprising the dual EGF-R/erbB-2 inhibitor: N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, pharmaceutical compositions comprising the same, and methods of using such combinations in the treatment of cancer.

### BACKGROUND OF THE INVENTION

Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

Protein tyrosine kinases (PTKs) catalyze the phosphorylation of specific tyrosyl residues in various proteins involved in the regulation of cell growth and differentiation. (A. F. Wilks, Progress in Growth Factor Research, 1990, 2, 97-111; S. A. Courtneidge, Dev. Supp. I, 1993, 57-64; J. A. Cooper, Semin. Cell Biol., 1994, 5(6), 377-387; R. F. Paulson, Semin. Immunol., 1995, 7(4), 267-277; A. C. Chan, Curr. Opin. Immunol., 1996, 8(3), 394-401). Inappropriate or uncontrolled activation of many PTKs, i.e. aberrant PTK activity, for example by over-expression or mutation, has been shown to result in uncontrolled cell growth.

Aberrant protein tyrosine kinase (PTK) activity has been implicated in a variety of disorders including psoriasis, rheumatoid arthritis, bronchitis, as well as cancer. Development of effective treatments for such disorders is a constant and ongoing enterprise in the medical field. The ErbB family of PTKs, which includes ErbB-2, EGFR, ErbB-3 and ErbB-4, is one group of PTKs that has attracted interest as a therapeutic target. Currently, of special interest, is the role of ErbB family PTKs in hyperproliferative disorders, particularly human malignancies. Elevated EGFR activity has, for example, been implicated in non-small cell lung, bladder, and head and neck cancers. Furthermore, increased ErbB-2 activity has been implicated in breast, ovarian, gastric and pancreatic cancers. Overexpression of HRG and/or HER3 has been reported in numerous cancers, including gastric, ovarian, prostate, bladder, and breast tumors and is associated with poor prognosis (B. Tanner, J Clin Oncol. 2006, 24(26):4317-23; M. Hayashi, Clin. Cancer Res. 2008.14(23):7843-9; H. Kaya, Eur J Gynaecol Oncol. 2008; 29(4):350-6;). Consequently, inhibition of ErbB family PTKs should provide a treatment for disorders characterized by aberrant ErbB family PTK activity. The biological role of ErbB family PTKs and their implication in various disease states is discussed, for instance in U.S. Pat. No. 5,773,476; International Patent Application WO 99/35146; M. C. Hung et al, Seminars in Oncology, 26: 4, Suppl. 12 (August) 1999, 51-59; Ullrich et al, Cell, 61: 203-212, Apr. 20, 1990; Modjtahedi et al, Int'l J. of Oncology, 13: 335-342, 1998; and J. R. Woodburn, Pharmacol. Ther., 82: 2-3, 241-250, 1999, it is generally accepted that inhibitors of ErbB family kinases will be useful for the treatment of such cancers or other condition associated with ErbB family kinases.

Apoptosis (programmed cell death) plays essential roles in embryonic development and pathogenesis of various diseases, such as degenerative neuronal diseases, cardiovascular diseases and cancer. Recent work has led to the identification of various pro- and anti-apoptotic gene products that are involved in the regulation or execution of programmed cell death. Expression of anti-apoptotic genes, such as Bcl2 or Bcl-x_{L}, inhibits apoptotic cell death induced by various stimuli. On the other hand, expression of pro-apoptotic genes, such as Bax or Bad, leads to programmed cell death (Adams et al. Science, 281:1322-1326 (1998)). The execution of programmed cell death is mediated by caspase-1 related proteinases, including caspase-3, caspase-7, caspase-8 and caspase-9 etc (Thornberry et al. Science, 281:1312-1316 (1998)).

The phosphatidylinositol 3′-OH kinase (PI3K)/Akt/PKB pathway appears important for regulating cell survival/cell death (Kulik et al. Mol. Cell. Biol. 17:1595-1606 (1997); Franke et al, Cell, 88:435-437 (1997); Kauffmann-Zeh et al. Nature 385:544-548 (1997) Hemmings Science, 275:628-630 (1997); Dudek et al., Science, 275:661-665 (1997)). Survival factors, such as platelet derived growth factor (PDGF), nerve growth factor (NGF) and insulin-like growth factor-1 (IGF-I), promote cell survival under various conditions by inducing the activity of PI3K (Kulik et al. 1997, Hemmings 1997). Activated PI3K leads to the production of phosphatidylinositol (3,4,5)-triphosphate (Ptdlns (3,4,5)-P3), which in turn binds to, and promotes the activation of, the serine/threonine kinase Akt, which contains a pleckstrin homology (PH)-domain (Franke et al Cell, 81:727-736 (1995); Hemmings Science, 277:534 (1997); Downward, Curr. Opin. Cell Biol. 10:262-267 (1998), Alessi et al., EMBO J. 15: 6541-6551 (1996)). Specific inhibitors of PI3K or dominant negative Akt/PKB mutants abolish survival-promoting activities of these growth factors or cytokines. It has been previously disclosed that inhibitors of PI3K (LY294002 or wortmannin) blocked the activation of Akt/PKB by upstream kinases. In addition, introduction of constitutively active PI3K or Akt/PKB mutants promotes cell survival under conditions in which cells normally undergo apoptotic cell death (Kulik et al. 1997, Dudek et al. 1997).

The tumor suppressor PTEN, a protein and lipid phosphatase that specifically removes the 3′ phosphate of Ptdlns(3,4,5)—P3, is a negative regulator of the PI3K/Akt pathway (Li et al. Science 275:1943-1947 (1997), Stambolic et al. Cell 95:29-39 (1998), Sun et al. Proc. Natl. Acad. Sci. U.S.A. 96:6199-6204 (1999)). Germline mutations of PTEN are responsible for human cancer syndromes such as Cowden disease (Liaw et al. Nature Genetics 16:64-67 (1997)). PTEN is deleted in a large percentage of human tumors and tumor cell lines without functional PTEN show elevated levels of activated Akt (Li et al. supra, Guldberg et al. Cancer Research 57:3660-3663 (1997), Risinger et al. Cancer Research 57:4736-4738 (1997)).

These observations demonstrate that the PI3K/Akt pathway plays important roles for regulating cell survival or apoptosis in tumorigenesis and/or cancer.

It would be useful to provide a novel therapy which provides more effective and/or enhanced treatment of an individual suffering the effects of cancer.

### SUMMARY OF THE INVENTION

One embodiment of this invention provides a combination comprising:

(i) a compound of Structure (I):

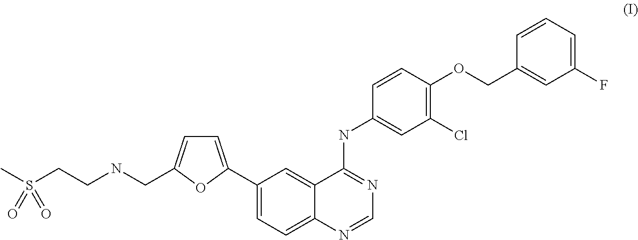 [SMILES: CS(=O)(=O)CCNCc1ccc(-c2ccc3ncnc(Nc4ccc(OCc5cccc(F)c5)c(Cl)c4)c3c2)o1]

or a pharmaceutically acceptable hydrate and/or salt thereof; and

(ii) a compound of Structure (II):

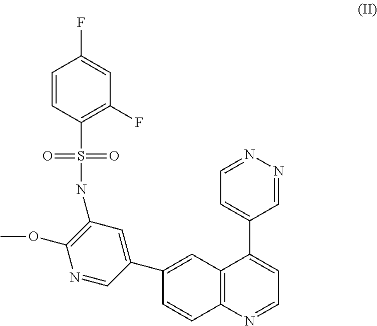 [SMILES: COc1ncc(-c2ccc3nccc(-c4ccnnc4)c3c2)cc1NS(=O)(=O)c1ccc(F)cc1F]

2,4-difluoro-N-{2-(methyloxy)-5-[4-(4-pyridazinyl)-6-quinolinyl]-3-pyridinyl}benzenesulfonamide or a pharmaceutically acceptable salt thereof.

One embodiment of this invention provides a method of treating cancer in a human in need thereof which comprises the in vivo administration of a therapeutically effective amount of a combination of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and 2,4-difluoro-N-{2-(methyloxy)-5-[4-(4-pyridazinyl)-6-quinolinyl]-3-pyridinyl}benzenesulfonamide or a pharmaceutically acceptable salt thereof, to such human.

One embodiment of this invention provides a method of treating cancer in a human in need thereof which comprises the in vivo administration of a therapeutically effective amount of a combination of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt thereof, and 2,4-difluoro-N-{2-(methyloxy)-5-[4-(4-pyridazinyl)-6-quinolinyl]-3-pyridinyl}benzenesulfonamide or a pharmaceutically acceptable salt thereof to such human,

wherein the combination is administered within a specified period, and

wherein the combination is administered for a duration of time.

One embodiment of this invention provides a method of treating cancer in a human in need thereof which comprises the in vivo administration of a therapeutically effective amount of a combination of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and 2,4-difluoro-N-{2-(methyloxy)-5-[4-(4-pyridazinyl)-6-quinolinyl]-3-pyridinyl}benzenesulfonamide or a pharmaceutically acceptable salt thereof to such human,

wherein the compounds of the combination are administered sequentially.

### DETAILED DESCRIPTION OF THE INVENTION

The present invention relates to combinations that exhibit antiproliferative activity. Suitably, the method relates to methods of treating cancer by the co-administration of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, (hereinafter Compound A, or a pharmaceutically acceptable hydrate and/or salt, suitably the ditosylate monohydrate salt, thereof,

which compound is represented by Structure I:

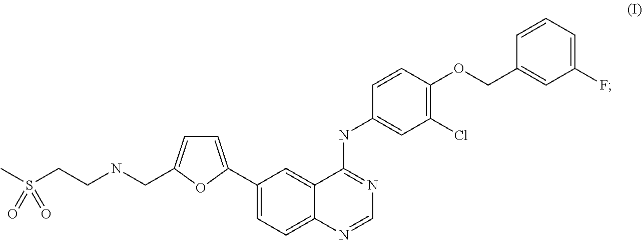 [SMILES: CS(=O)(=O)CCNCc1ccc(-c2ccc3ncnc(Nc4ccc(OCc5cccc(F)c5)c(Cl)c4)c3c2)o1]

and 2,4-difluoro-N-{2-(methyloxy)-5-[4-(4-pyridazinyl)-6-quinolinyl]-3-pyridinyl}benzenesulfonamide or a pharmaceutically acceptable salt thereof, (hereinafter Compound B or a pharmaceutically acceptable salt thereof,

which compound is represented by Structure II:

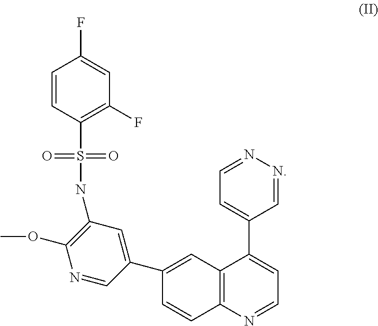 [SMILES: COc1ncc(-c2ccc3nccc(-c4ccnnc4)c3c2)cc1NS(=O)(=O)c1ccc(F)cc1F]

Compound A is disclosed and claimed, along with pharmaceutically acceptable solvates and salts thereof, as being useful as an inhibitor of erbB-2 activity, particularly in treatment of cancer, in International Application No. PCT/EP99/00048, having an International filing date of Jan. 8, 1999, International Publication Number WO 99/35146 and an International Publication date of Jul. 15, 1999, the entire disclosure of which is hereby incorporated by reference, Compound A is the compound of Example 29. Compound A can be prepared as described in International Application No. PCT/EP99/00048.

Suitably, Compound A is in the form of a ditosylate monohydrate salt. This salt form can be prepared by one of skill in the art from the description in International Application No. PCT/US01/20706, having an International filing date of Jun. 28, 2001, International Publication Number WO 02/02552 and an International Publication date of Jan. 10, 2002, the entire disclosure of which is hereby incorporated by reference, see particularly Example 10.

Suitable pharmaceutical compositions containing Compound A as a single active ingredient are prepared as described in International Application No. PCT/US2006/014447, having an International filing date of Apr. 18, 2006, International Publication Number WO 06/113649 and an International Publication date of Oct. 26, 2006, the entire disclosure of which is hereby incorporated by reference, see particularly the formulation in Table 3.

Compound A is sold commercially as the ditosylate monohydrate salt and is known by the generic name lapatinib and trade names Tykerb® and Tyverb®.

Compound B is disclosed and claimed, along with pharmaceutically acceptable salts thereof, as being useful as an inhibitor of PI3K activity, particularly in treatment of cancer, in International Application No. PCT/US2008/063819, having an International filing date of May 16, 2008; International Publication Number WO 2008/1444463 and an International Publication date of Nov. 27, 2008, the entire disclosure of which is hereby incorporated by reference, Compound B is the compound of example 345. Compound B can be prepared as described in International Application No. PCT/US2008/063819.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect proflie, v) an increase in the therapeutic window, or vi) an increase in the bioavailability of one or both of the component compounds.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable hydrates and/or salts thereof, and Compound B, and pharmaceutically acceptable salts thereof.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Suitably the solvent used is water.

The pharmaceutically acceptable salts of the compounds of the invention are readily prepared by those of skill in the art.

Also, contemplated herein is a method of treating cancer using a combination of the invention where Compound A, or a pharmaceutically acceptable hydrate and/or salt thereof, and/or Compound B or a pharmaceutically acceptable salt thereof are administered as pro-drugs. Pharmaceutically acceptable pro-drugs of the compounds of the invention are readily prepared by those of skill in the art.

When referring to a dosing protocol, the term “day”, “per day” and the like, refer to a time within one calendar day which begins at midnight and ends at the following midnight.

By the term “treating” and derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and derivatives thereof, as used herein is meant either, simultaneous administration or any manner of separate sequential administration of a therapeutically effective amount of Compound A, or a pharmaceutically acceptable hydrate and/or salt thereof, and Compound B or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable hydrate and/or salt thereof, and Compound B, or a pharmaceutically acceptable salt thereof, according to the invention. When both compounds are administered simultaneously, the combination kit can contain Compound A, or a pharmaceutically acceptable hydrate and/or salt thereof, and Compound B, or a pharmaceutically acceptable salt thereof, in a single pharmaceutical composition, such as a tablet, or in separate pharmaceutical compositions. When the compounds are not administered simultaneously, the combination kit will contain Compound A, or a pharmaceutically acceptable hydrate and/or salt thereof, and Compound B, or a pharmaceutically acceptable salt thereof, in separate pharmaceutical compositions. The combination kit can comprise Compound A, or a pharmaceutically acceptable hydrate and/or salt thereof, and Compound B, or a pharmaceutically acceptable salt thereof, in separate pharmaceutical compositions in a single package or in separate pharmaceutical compositions in separate packages.

In one aspect there is provided a combination kit comprising the components:

Compound A, or a pharmaceutically acceptable hydrate and/or salt thereof, in association with a pharmaceutically acceptable carrier; and

Compound B, or a pharmaceutically acceptable salt thereof, in association with a pharmaceutically acceptable carrier.

In one embodiment of the invention the combination kit comprises the following components:

Compound A, or a pharmaceutically acceptable hydrate and/or salt thereof, in association with a pharmaceutically acceptable carrier; and

Compound B, or a pharmaceutically acceptable salt thereof, in association with a pharmaceutically acceptable carrier,

wherein the components are provided in a form which is suitable for sequential, separate and/or simultaneous administration.

In one embodiment the combination kit comprises:

a first container comprising Compound A, or a pharmaceutically acceptable hydrate and/or salt thereof, in association with a pharmaceutically acceptable carrier; and

a second container comprising Compound B, or a pharmaceutically acceptable salt thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

As used herein the term “Compound A²” means—compound A, or a pharmaceutically acceptable hydrate and/or salt thereof—.

As used herein the term “Compound B²” means—Compound B, or a pharmaceutically acceptable salt thereof—.

Suitably the combinations of this invention are administered within a “specified period”.

By the term “specified period” and derivatives thereof, as used herein is meant the interval of time between the administration of one of Compound A² and Compound B² and the other of Compound A² and Compound B². Unless otherwise defined, the specified period can include simultaneous administration. When both compounds of the invention are administered once a day the specified period refers to timing of the administration of Compound A² and Compound B² during a single day. When one or both compounds of the invention are administered more than once a day, the specified period is calculated based on the first administration of each compound on a specific day. All administrations of a compound of the invention that are subsequent to the first during a specific day are not considered when calculating the specific period.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and derivatives thereof, as used herein is meant that both compounds of the invention are administered within a “specified period” for an indicated number of consecutive days, optionally followed by a number of consecutive days where only one of the component compounds is administered. Unless otherwise defined, the “duration of time” and in all dosing protocols described herein, do not have to commence with the start of treatment and terminate with the end of treatment, it is only required that the number of consecutive days in which both compounds are administered and the optional number of consecutive days in which only one of the component compounds is administered, or the indicated dosing protocol, occur at some point during the course of treatment.

Regarding “specified period” administration:

Suitably, during the course of treatment, both compounds will be administered within a specified period for at least 1 day—in this case, the duration of time will be at least 1 day; suitably, during the course of treatment, both compounds will be administered within a specified period for at least 2 consecutive days—in this case, the duration of time will be at least 2 days; suitably, during the course of treatment, both compounds will be administered within a specified period for at least 3 consecutive days—in this case, the duration of time will be at least 3 days; suitably, during the course of treatment, both compounds will be administered within a specified period for at least 5 consecutive days—in this case, the duration of time will be at least 5 days; suitably, during the course of treatment, both compounds will be administered within a specified period for at least 7 consecutive days—in this case, the duration of time will be at least 7 days; suitably, during the course of treatment, both compounds will be administered within a specified period for at least 14 consecutive days—in this case, the duration of time will be at least 14 days; suitably, during the course of treatment, both compounds will be administered within a specified period for at least 30 consecutive days—in this case, the duration of time will be at least 30 days. When, during the course of treatment, both compounds are administered within a specified period for over 30 days, the treatment is considered chronic treatment and will continue until an altering event, such as a reassessment in cancer status or a change in the condition of the patient, warrants a modification to the protocol.

Further regarding “specified period” administration:

Suitably, during the course of treatment, both compounds will be administered within a specified period for at least 1 day, followed by the administration of Compound A² alone for at least 1 day—in this case, the duration of time will be at least 2 days; suitably, during the course of treatment, both compounds will be administered within a specified period for at least 1 day, followed by administration of Compound A² alone for at least 2 days—in this case, the duration of time will be at least 3 days; suitably, during the course of treatment, both compounds will be administered within a specified period for at least 1 day, followed by administration of Compound A² alone for at least 3 days—in this case, the duration of time will be at least 4 days; suitably, during the course of treatment, both compounds will be administered within a specified period for at least 1 day, followed by administration of Compound A² alone for at least 4 days—in this case, the duration of time will be at least 5 days; suitably, during the course of treatment, both compounds will be administered within a specified period for at least 1 day, followed by administration of Compound A² alone for at least 5 days—in this case, the duration of time will be at least 6 days; suitably, during the course of treatment, both compounds will be administered within a specified period for at least 1 day, followed by administration of Compound A² alone for at least 6 days—in this case, the duration of time will be at least 7 days; suitably, during the course of treatment, both compounds will be administered within a specified period for at least 1 day, followed by administration of Compound A² alone for at least 7 days—in this case, the duration of time will be at least 8 days; suitably, during the course of treatment, both compounds will be administered within a specified period for at least 2 consecutive days, followed by administration of Compound A² alone for at least 1 day—in this case, the duration of time will be at least 3 days; suitably, during the course of treatment, both compounds will be administered within a specified period for at least 2 consecutive days, followed by administration of Compound A² alone for at least 2 consecutive days—in this case, the duration of time will be at least 4 days; suitably, during the course of treatment, both compounds will be administered within a specified period for at least 2 consecutive days, followed by administration of Compound A² alone for at least 3 consecutive days—in this case, the duration of time will be at least 5 days; suitably, during the course of treatment, both compounds will be administered within a specified period for at least 2 consecutive days, followed by administration of Compound A² alone for at least 4 consecutive days—in this case, the duration of time will be at least 6 days; suitably, during the course of treatment, both compounds will be administered within a specified period for at least 2 consecutive days, followed by administration of Compound A² alone for at least 5 consecutive days—in this case, the duration of time will be at least 7 days; suitably, during the course of treatment, both compounds will be administered within a specified period for at least 2 consecutive days, followed by administration of Compound A² alone for at least 6 consecutive days—in this case, the duration of time will be at least 8 days; suitably, during the course of treatment, both compounds will be administered within a specified period for at least 2 consecutive days, followed by administration of Compound A² alone for at least 7 consecutive days—in this case, the duration of time will be at least 9 days; suitably, during the course of treatment, both compounds will be administered within a specified period for at least 3 consecutive days, followed by administration of Compound A² alone for at least 1 day—in this case, the duration of time will be at least 4 days; suitably, during the course of treatment, both compounds will be administered within a specified period for at least 3 consecutive days, followed by administration of Compound A² alone for at least 2 consecutive days—in this case, the duration of time will be at least 5 days; suitably, during the course of treatment, both compounds will be administered within a specified period for at least 3 consecutive days, followed by administration of Compound A² alone for at least 3 consecutive days—in this case, the duration of time will be at least 6 days; suitably, during the course of treatment, both compounds will be administered within a specified period for at least 3 consecutive days, followed by administration of Compound A² alone for at least 4 consecutive days—in this case, the duration of time will be at least 7 days; suitably, during the course of treatment, both compounds will be administered within a specified period for at least 3 consecutive days, followed by administration of Compound A² alone for at least 5 consecutive days—in this case, the duration of time will be at least 8 days; suitably, during the course of treatment, both compounds will be administered within a specified period for at least 3 consecutive days, followed by administration of Compound A² alone for at least 6 consecutive days—in this case, the duration of time will be at least 9 days; suitably, during the course of treatment, both compounds will be administered within a specified period for at least 3 consecutive days, followed by administration of Compound A² alone for at least 7 consecutive days—in this case, the duration of time will be at least 10 days; suitably, during the course of treatment, both compounds will be administered within a specified period for at least 4 consecutive days, followed by administration of Compound A² alone for at least 1 day—in this case, the duration of time will be at least 5 consecutive days; suitably, during the course of treatment, both compounds will be administered within a specified period for at least 4 consecutive days, followed by administration of Compound A² alone for at least 2 consecutive days—in this case, the duration of time will be at least 6 consecutive days; suitably, during the course of treatment, both compounds will be administered within a specified period for at least 4 consecutive days, followed by administration of Compound A² alone for at least 3 consecutive days—in this case, the duration of time will be at least 7 consecutive days; suitably, during the course of treatment, both compounds will be administered within a specified period for at least 4 consecutive days, followed by administration of Compound A² alone for at least 4 consecutive days—in this case, the duration of time will be at least 8 consecutive days; suitably, during the course of treatment, both compounds will be administered within a specified period for at least 4 consecutive days, followed by administration of Compound A² alone for at least 7 consecutive days—in this case, the duration of time will be at least 11 consecutive days; suitably, during the course of treatment, both compounds will be administered within a specified period for at least 5 consecutive days, followed by administration of Compound A² alone for at least 1 day—in this case, the duration of time will be at least 6 consecutive days; suitably, during the course of treatment, both compounds will be administered within a specified period for at least 5 consecutive days, followed by administration of Compound A² alone for at least 2 consecutive days—in this case, the duration of time will be at least 7 consecutive days; suitably, during the course of treatment, both compounds will be administered within a specified period for at least 5 consecutive days, followed by administration of Compound A² alone for at least 3 consecutive days—in this case, the duration of time will be at least 8 consecutive days; suitably, during the course of treatment, both compounds will be administered within a specified period for at least 5 consecutive days, followed by administration of Compound A² alone for at least 4 consecutive days—in this case, the duration of time will be at least 9 consecutive days; suitably, during the course of treatment, both compounds will be administered within a specified period for at least 5 consecutive days, followed by administration of Compound A² alone for at least 5 consecutive days—in this case, the duration of time will be at least 10 consecutive days; suitably, during the course of treatment, both compounds will be administered within a specified period for at least 7 consecutive days, followed by administration of Compound A² alone for at least 2 consecutive days—in this case, the duration of time will be at least 9 consecutive days; suitably, during the course of treatment, both compounds will be administered within a specified period for at least 14 consecutive days, followed by administration of Compound A² alone for at least 7 consecutive days—in this case, the duration of time will be at least 21 consecutive days; suitably, during the course of treatment, both compounds will be administered within a specified period for at least 30 consecutive days, followed by administration of Compound A² alone for at least 7 consecutive days—in this case, the duration of time will be at least 37 consecutive days. Suitably, during the course of treatment, both compounds will be administered within a specified period for from 1 to 3 consecutive days, followed by administration of Compound A² alone for from 3 to 7 consecutive days. Suitably, during the course of treatment, both compounds will be administered within a specified period for from 3 to 6 consecutive days, followed by administration of Compound A² alone for from 1 to 4 consecutive days. Suitably, during the course of treatment, both compounds will be administered within a specified period for 5 consecutive days, followed by administration of Compound A² alone for 2 consecutive days. Suitably, during the course of treatment, both compounds will be administered within a specified period for 2 consecutive days, followed by administration of Compound A² alone for from 3 to 7 consecutive days. Suitably, during the course of treatment, both compounds will be administered within a specified period for from 1 to 3 days over a 7 day period, and during the other days of the 7 day period Compound A² will be administered alone.

Suitably, during the course of treatment, both compounds will be administered within a specified period for 2 days over a 7 day period, and during the other days of the 7 day period Compound A² will be administered alone.

Suitably, if the compounds are not administered during a “specified period”, they are administered sequentially. By the term “sequential administration”, and derivates thereof, as used herein is meant that one of Compound A² and Compound B² is administered for one or more consecutive days and the other of Compound A² and Compound B² is subsequently administered for one or more consecutive days. Unless otherwise defined, the “sequential administration” and in all dosing protocols described herein, do not have to commence with the start of treatment and terminate with the end of treatment, it is only required that the administration of one of Compound A² and Compound B² followed by the administration of the other of Compound A² and Compound B², or the indicated dosing protocol, occur at some point during the course of treatment. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding Sequential Administration:

Suitably, one of Compound A² and Compound B² is administered for from 1 to 30 consecutive days, followed by an optional drug holiday, followed by administration of the other of Compound A² and Compound B² for from 1 to 30 consecutive days. Suitably, one of Compound A² and Compound B² is administered for from 1 to 21 consecutive days, followed by an optional drug holiday, followed by administration of the other of Compound A² and Compound B² for from 1 to 21 consecutive days. Suitably, one of Compound A² and Compound B² is administered for from 1 to 14 consecutive days, followed by a drug holiday of from 1 to 14 days, followed by administration of the other of Compound A² and Compound B² for from 1 to 14 consecutive days. Suitably, one of Compound A² and Compound B² is administered for from 2 to 7 consecutive days, followed by a drug holiday of from 2 to 10 days, followed by administration of the other of Compound A² and Compound B² for from 2 to 7 consecutive days.

Suitably, Compound B² will be administered first in the sequence, followed by an optional drug holiday, followed by administration of Compound A². Suitably, Compound B² is administered for from 1 to 21 consecutive days, followed by an optional drug holiday, followed by administration of Compound A² for from 1 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

Suitably, Compound A² will be administered first in the sequence, followed by an optional drug holiday, followed by administration of Compound B². Suitably, Compound A² is administered for from 1 to 21 consecutive days, followed by an optional drug holiday, followed by administration of Compound B² for from 1 to 21 consecutive days. Suitably, Compound A² is administered for from 3 to 21 consecutive days, followed by a drug holiday of from 1 to 14 days, followed by administration of Compound B² for from 3 to 21 consecutive days. Suitably, Compound A² is administered for from 3 to 21 consecutive days, followed by a drug holiday of from 3 to 14 days, followed by administration of Compound B² for from 3 to 21 consecutive days. Suitably, Compound A² is administered for 21 consecutive days, followed by an optional drug holiday, followed by administration of Compound B² for 14 consecutive days. Suitably, Compound A² is administered for 14 consecutive days, followed by a drug holiday of from 1 to 14 days, followed by administration of Compound B² for 14 consecutive days. Suitably, Compound A² is administered for 7 consecutive days, followed by a drug holiday of from 3 to 10 days, followed by administration of Compound B² for 7 consecutive days. Suitably, Compound A² is administered for 3 consecutive days, followed by a drug holiday of from 3 to 14 days, followed by administration of Compound B² for 7 consecutive days. Suitably, Compound A² is administered for 3 consecutive days, followed by a drug holiday of from 3 to 10 days, followed by administration of Compound B² for 3 consecutive days. Suitably, Compound A² is administered for 7 consecutive days, followed by administration of Compound B² for 1 day. Suitably, Compound A² is administered for 6 consecutive days, followed by administration of Compound B² for 1 day. Suitably, Compound B² is administered for 1 day, followed by administration of Compound A² for 7 consecutive days. Suitably, Compound B² is administered for 1 day, followed by administration of Compound A² for 6 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 250 mg to about 1,500 mg; suitably, the amount will be selected from about 500 mg to about 1,250 mg; suitably, the amount will be selected from about 750 mg to about 1,000 mg; suitably, the amount will be 250 mg, suitably, the amount will be 500 mg, suitably, the amount will be 750 mg, suitably, the amount will be 1,000 mg, suitably, the amount will be 1,250 mg; suitably, the amount will be 1,500 mg. Accordingly, the amount of Compound A² administered as part of the combination according to the present invention will be an amount selected from about 250 mg to about 1,500 mg. For example, the amount of Compound A² administered as part of the combination according to the present invention is suitably selected from 250 mg, 500 mg, 750 mg, 1,000 mg, 1,250 mg and 1,500 mg. Suitably, the selected amount of Compound A² is administered from 1 to 4 times a day, in one or more tablets. Suitably, the selected amount of Compound A² is administered twice a day, in one or more tablets. Suitably, the selected amount of Compound A² is administered once a day, in one or more tablets.

Suitably, the amount of Compound B² administered as part of the combination according to the present invention will be an amount selected from about 0.25 mg to about 75 mg; suitably, the amount will be selected from about 0.5 mg to about 50 mg; suitably, the amount will be selected from about 1 mg to about 25 mg; suitably, the amount will be selected from about 2 mg to about 20 mg; suitably, the amount will be selected from about 4 mg to about 16 mg; suitably, the amount will be selected from about 6 mg to about 12 mg; suitably, the amount will be about 10 mg. Accordingly, the amount of Compound B² administered as part of the combination according to the present invention will be an amount selected from about 0.5 mg to about 50 mg. For example, the amount of Compound B² administered as part of the combination according to the present invention can be 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 20 mg, 21 mg, 22 mg, 23 mg, 25 mg, 26 mg, 27 mg, 28 mg, 29 mg, 30 mg, 35 mg, 40 mg, 45 mg, or 50 mg.

As used herein, all amounts specified for Compound A² and Compound B² are indicated as the administered amount of free or unsalted and unsolvated compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, suitably, may be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

Suitably, the present invention relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,

malignant lymphoma, hodgkins lymphoma, non-hodgkins lymphoma, lymphoblastic T cell lymphoma, Burkitt's lymphoma, follicular lymphoma,

neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the present invention relates to a method of treating or lessening the severity of a cancer that is either wild type or mutant for Raf and either wild type or mutant for PI3K/Pten. This includes patients wild type for both Raf and PI3K/PTEN, mutant for both Raf and PI3K/PTEN, mutant for Raf and wild type for PI3K/PTEN and wild type for Raf and mutant for PI3K/PTEN.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

Cancers that are either wild type or mutant for Raf and either wild type or mutant for PI3K/Pten are identified by known methods.

For example, wild type or mutant Raf or PI3K/PTEN tumor cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, Western blot or immunocyto chemistry.

This invention provides a combination comprising N-{3-Chloro-4-[(3-fluorobenzyl)oxy]phenyl}-6-[5-({[2-(methanesulphony)ethyl]amino}methyl)-2-furyl]-4-quinazolinamine, or a pharmaceutically acceptable hydrate and/or salt thereof, suitably the ditosylate monohydrate salt thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides for a combination comprising N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof for use in therapy.

This invention also provides for a combination comprising N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 22,4-difluoro-N-{2-(methyloxy)-5-[4-(4-pyridazinyl)-6-quinolinyl]-3-pyridinyl}benzenesulfonamide, or a pharmaceutically acceptable salt thereof, for use in treating cancer.

This invention also provides a pharmaceutical composition comprising a combination of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides a combination kit comprising N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides for the use of a combination comprising N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, in the manufacture of a medicament. This invention also provides for the use of a combination comprising N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, in the manufacture of a medicament to treat cancer.

This invention also provides a method of treating cancer which comprises administering a combination of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, suitably the ditosylate monohydrate salt, thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, to a subject in need thereof.

The following examples are intended for illustration only and are not intended to limit the scope of the invention in any way.

### Experimental Details

Preparation of Compounds:

Compound A is disclosed and claimed, along with pharmaceutically acceptable solvates and salts thereof, as being useful as an inhibitor of erbB-2 activity, particularly in treatment of cancer, in International Application No. PCT/EP99/00048, having an International filing date of Jan. 8, 1999, International Publication Number WO 99/35146 and an International Publication date of Jul. 15, 1999, the entire disclosure of which is hereby incorporated by reference, Compound A is the compound of Example 29. Compound A can be prepared as described in International Application No. PCT/EP99/00048.

Suitably, Compound A is in the form of a ditosylate monohydrate salt. This salt form can be prepared by one of skill in the art from the description in International Application No. PCT/US01/20706, having an International filing date of Jun. 28, 2001, International Publication Number WO 02/02552 and an International Publication date of Jan. 10, 2002, the entire disclosure of which is hereby incorporated by reference, see particularly Example 10.

Compound B is disclosed and claimed, along with pharmaceutically acceptable salts thereof, as being useful as an inhibitor of PI3K activity, particularly in treatment of cancer, in International Application No. PCT/US2008/063819, having an International filing date of May 16, 2008; International Publication Number WO 2008/1444463 and an International Publication date of Nov. 27, 2008, the entire disclosure of which is hereby incorporated by reference, Compound B is the compound of example 345. Compound B can be prepared as described in International Application No. PCT/US2008/063819

Methods:

Cell Lines and Growth Conditions

Human tumor cell lines from breast, HCC2218, HCC1419, BT-474, SK-BR-3, UACC893, JIMT-1, MDA-MB-361, HCC202, MDA-MB-175-VII, MDA-MB-453, MDA-MB-415, HCC1569, HCC1937, HCC38, MDA-MB-157, HCC1806 and ZR-75-1; colon, NCI-HS08; lung, CALU-3; melanoma, CHL-1; and prostate, BxPC3, cultured in RPMI 1640 medium containing 10% FBS; SKBR3-W13 and BT-474-J4 cultured in RPMI 1640 medium containing 10% FBS and 1 μM Compound A (in this assay, Compound A, was used in the form of the ditosylate monohydrate salt lapatinib); and KPL4 cultured in DMEM containing 5% FBS were kept in a humidified incubator at 37° C. in 95% air and 5% CO₂. JIMT-1 is a line derived from a patient clinically resistant to trastuzumab (Herceptin®). SK-BR-3-W13 is a single cell clone isolated by a cloning cylinder after a single treatment of SK-BR-3 cells with 0.5 μM lapatinib. BT-474-J4 is a single cell clone derived from a culture of BT-474 cells that were selected to grow in lapatinib to a concentration of 3 μM.

Cell Growth Inhibition Assay and Combination Data Analysis.

All cells were cultured without lapatinib for a minimum of 72 hours prior to cell plating. Cells were assayed in a 96-well tissue culture plate (NUNC 136102) of RPMI medium containing 10

% FBS for all cells at 2,000 cells per well except KPL4, which were plated in DMEM containing 5% FBS at 500 cells per well. Approximately 24 hours after plating, cells were exposed to ten, two-fold or three-fold serial dilutions of compound or the combination of the two agents at a constant molar to molar ratio of 10:1 Compound A to Compound B in RPMI media containing 10% FBS or DMEM containing 5% FBS at 500 cells per well for KPL4. Cells were incubated in the presence of compounds for 3 days. ATP levels were determined by adding Cell Titer Glo® (Promega) according to the manufacturer's protocol. Briefly, Cell Titer Glo® was added each plate, incubated for 20 minutes then luminescent signal was read on the SpectraMax L plate reader with a 0.5 sec integration time.

Inhibition of cell growth was estimated after treatment with compound or combination of compounds for three days and comparing the signal to cells treated with vehicle (DMSO). Cell growth was calculated relative to vehicle (DMSO) treated control wells. Concentration of compound that inhibits 50% of control cell growth (IC₅₀) was interpolated using nonlinear regression with the equation, y=(A+(B−A)/(1+(C/x)̂D)))), where A is the minimum response (yₘᵢₙ), B is the maximum response (yₘₐₓ), C is the inflection point of the curve (EC₅₀) and D is the Hill coefficient.

Combination effects on potency were evaluated using Combination Index (CI) which was calculated with the back-interpolated IC₅₀ values and the mutually non-exclusive equation derived by Chou and Talalay (Chou T C, Talalay P. Quantitative analysis of dose-effect relationships: the combined effects of multiple drugs or enzyme inhibitors. Adv Enzyme Regul 1984; 22:27-55):

CI=Da/IC₅₀(a)+Db/IC₅₀(b)+(Da×Db)/(IC₅₀(a)×IC₅₀(b))

where IC₅₀(a) is the IC₅₀ of the inhibitor A; IC₅₀(b) is the IC₅₀ for the inhibitor B; Da is the concentration of the inhibitor A in combination with the inhibitor B that inhibited 50% of cell growth; and Db is the concentration of inhibitor B in combination with the inhibitor A that inhibited 50% of cell growth. In general, a CI value <0.9, between 0.9 and 1.1, or >1.1 indicates synergy, additivity and antagonism, respectively. In general, the smaller the CI number, the greater is the strength of synergy.

The combination effects on the response scale were quantified by Excess Over Highest Single Agent (EOHSA). EOHSA values are defined as increases in improvement (here, in ‘percentage points’ (ppts) difference) produced by the combination over the best single drug at its component dose level. More specifically, suppose we have a combination composed of drug 1 at dose d1 and drug 2 at dose d2. If the effect of the combination of drugs 1 and 2 at doses d1 and d2 is better than either drug 1 (alone) at dose d1 or drug 2 (alone) at dose d2, then the combination is said to have a positive EOHSA and beneficial for that combination. For a combination drug experiment (involving drug 1 at dose d1 and drug 2 at dose d2), a drug combination (at total dose d1+d2) is said to have a statistically significant EOHSA if the mean response at the combination is significantly better than the mean responses for either drug 1 (alone) at dose d1 or drug 2 (alone) at dose d2. EOHSA is a common approach for evaluating drug combinations, and is an FDA criterion (21 CRF 300.50) for combination drug approval. See Borisy et al. (Borisy A A, Elliott P J, Hurst N R, Lee M S, Lehar J, Price E R, et al. Systematic discovery of multicomponent therapeutics. Proc Natl Acad Sci USA 2003 Jun. 24; 100(13):7977-82) or Hung et al. (Hung H M, Chi G Y, Lipicky R J. Testing for the existence of a desirable dose combination. Biometrics 1993 March; 49(1):85-94) for examples and discussion. The EOHSA analysis was conducted as follows. Since dose response curves were fit to the experimental data (for both of the single drug regimens and also for the combination drug at a fixed-dose-ratio ray), comparisons needed for making EOHSA statistical inferences could be done by interpolation using the fitted regression models. At specified total dose levels of IC50 along the dose response curve of a fixed-dose-ratio ray, the dose combination (corresponding to IC50,) was determined for making EOHSA statistical inferences. Here, the mean response at a given combination, IC50 for example, was compared to the mean response at the component dose levels for drugs 1 and 2 on their dose response curves. More specifically, suppose that the IC50 for the combination drug (along the fixed-dose-ratio ray) corresponds to a total dose of d1+d2. We then compare the mean response for the combination (d1+d2) to drug 1 at d1 and drug 2 at d2 using the respective fitted dose response curves corresponding to the fixed—dose-ratio combination curve and the dose response curves for drugs 1 and 2 alone.

Cell Apoptosis Assays-DNA Fragmentation and Caspase-3/7 Activation.

For investigation of the induction of apoptosis, cells were plated at 5,000 cells per well in a 96-well tissue culture plate and allowed to attach for approximately 24 hours. Cells were then treated with compounds as described above. 24 hours after compound treatment, the levels of active caspase 3 and caspase 7 were determined with the Caspase Glo™ 3/7 (Promega, cat G8093) according to the instructions provided by the manufacturer. 48 hours after treatment with compound, levels of apoptosis were estimated using the Roche Cell Death ELISA (Cat. No. 11 774 425 001) following the instructions provided by the manufacturer.

### BRIEF DESCRIPTION OF THE DRAWINGS

FIG. 1. Cell growth inhibition (A.) and caspase 3/7 activity (B.) were determined as described in the methods 72 h and 24 h, respectively, post treatment of BT474 and UACC893 cells with Compound A, Compound B or the 10 to 1 molar ratio combination of Compound A and Compound B

### RESULTS

As used in the “results section”, Compound A is meant N-{3-Chloro-4-[(3-fluorobenzyl)oxy]phenyl}-6-[5-({[2-(methanesulphonyl)ethyl]amino}methyl)-2-furyl]-4-quinazolinamine ditosylate monohydrate.

As used in the “results section”, Compound B is meant the 2,4-difluoro-N-{2-(methyloxy)-5-[4-(4-pyridazinyl)-6-quinolinyl]-3-pyridinyl}benzenesulfonamide.

Cell Growth Inhibition by Compound A, Compound B and the Combination of Compound A with Compound B.

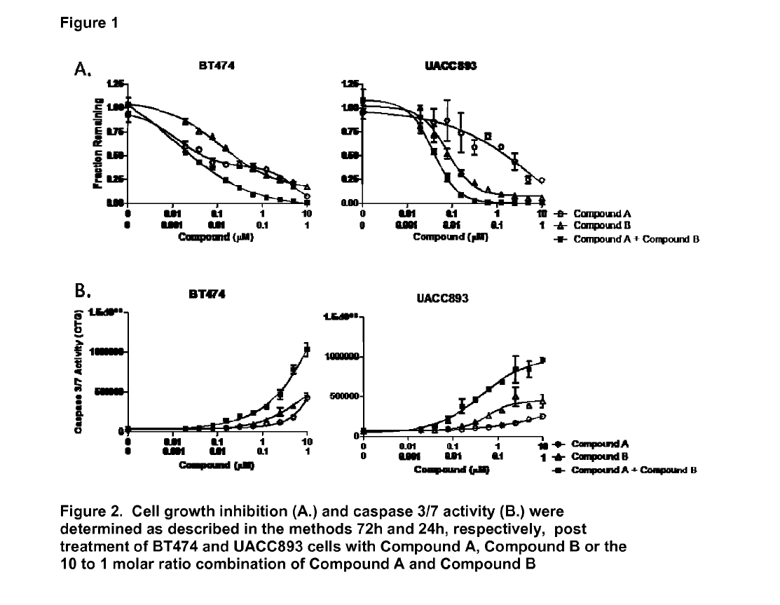

The effects of cell growth inhibition by Compound A, Compound B and their combination were determined in twelve HER2 positive (HER2+) breast tumor lines, HCC2218, UACC893, MDA-MB-453, KPL-4, MDA-MB-361, HCC202, HCC1419, BT474, SK-BR-3, BT474-J4, SK-BR-3-W13 and JIMT-1; eight HER2 negative (HER2−) breast tumor lines, MDA-MB-175-VII-MDA-MB-415, HCC1569, HCC1937, HCC38, MDA-MB-157, HCC1806 and ZR-75-1; the colon cell line, NCI-HS08; the HER2+ lung cell line, CALU-3; and the melanoma cell line, CHL-1. The mean IC₅₀s (from at least two independent experiments) and the combination effects at IC₅₀s are summarized in Table 1. Representative dose response curves for BT474 and UACC893 cells are provided in FIG. 1A.

HCC2218, HCC1419, BT474 and SK-BR-3 HER2+ lines and MDA-MB-175-VII HER2− line are highly sensitive to Compound A with IC₅₀ values of less than 0.2 μM and to Compound B with IC₅₀<0.05 μM. The combination of Compound A and Compound B showed synergy with combination index (CI) values between 0.3 and 0.9 and greater than the most active single agent analysis (EOSHA) between 13 and 35 ppts.

UACC893 and KPL-4 HER2+ lines with an H1047R PIK3CA mutation are sensitive to both Compound A with IC₅₀ values less than 1 μM and Compound B with IC₅₀ values less than 0.005 μM as single agents. The combination of Compound A and Compound B showed synergistic effects as demonstrated by the CI values, 0.67 and 0.50, respectively, and EOHSA values (23 and 28 ppt respectively). MDA-MB-453, a HER2+ cell line with an H1047R PIK3CA mutation, MDA-MB-361, a HER2+ cell line with an E545K PIK3CA mutation, and HCC202, a HER2+ cell line with an E545K PIK3CA mutation, are less sensitive to Compound A. MDA-MB-453 and MDA-MB-361 cells are sensitive to Compound B with IC₅₀ values of 0.015 and 0.009 μM, respectively. HCC202 cells are less sensitive to Compound B with IC₅₀ value of 0.2 μM. The combination of Compound A and Compound B is beneficial in MDA-MB-361 as indicated by the EOSHA value of 8 ppts and is synergistic in MDA-MB-453 and HCC202 as indicated by the CI values of 0.45 and 0.23 and EOHSA values of 25 and 22 ppts, respectively.

Both BT474-J4 and SK-BR-3-W13 lines are HER2+, Compound A acquired resistant clones developed from BT474 and Sk-Br-3 cells respectively. JIMT-1 is a line derived from a patient who was resistant to trastuzumab therapy (Tanner et al, Mol Cancer Ther 2004; 3:1585-92). BT474-J4 and JIMT-1 cell lines are sensitive to cell growth inhibition by Compound B with IC₅₀ values less than 0.03 μM. The combination of Compound A and Compound B is synergistic in BT474-J4 cells with the CI value of 0.31 and EOSHA value of 20 ppts. The combination of Compound A and Compound B in SK-BR-3-W13 and JIMT-1 cell lines showed modest synergy with CI values of 0.55 and 0.71, respectively, and a greater effect than the most active single agent of 9 and 8 ppts, respectively.

The additional breast cell lines, MDA-MB-415, HCC1569, HCC1937, HCC38, MDA-MB-157, HCC1806 and ZR-75-1, were not sensitive to cell growth inhibition by Compound A (IC₅₀ values >1 μM) and displayed a range of sensitivities to Compound B (IC₅₀ values from 0.01 to 1.3 μM. The combination of Compound A and Compound B was synergistic in MDA-MB-453 and HCC1937 cells with CI values of 0.45 and 0.61, respectively. The MDA-MB-415, HCC1569, HCC38, MDA-MB-157, HCC1806 and ZR-75-1 cell lines displayed a benefit in cell growth inhibition with the combination of Compound A and Compound B as determined by the EOHSA range of 4 to 18 ppts.

NCI-HS08, CALU-3 and CHL-1 cells are sensitive to cell growth inhibition by Compound A (IC₅₀ values less than 0.4 μM) and Compound B (IC₅₀ values less than 0.04 μM) as single agents. The combination of Compound A and Compound B was synergistic in these cells with CI values of 0.58, 0.48 and 0.51, and EOHSA values of 18, 27 and 28 ppts, respectively.

Increase of Caspase Activity by Compound A, Compound B and their Combination in Tumor Cell Lines

A subset of HER2+ breast cell lines, BT474, HCC1419 and UACC893 cells were further evaluated for the ability of Compound A, Compound B or the combination of Compound A and Compound B to induce caspase 3/7 activity. Activation of caspase 3 is a hallmark of induction of apoptosis. Representative caspase 3/7 activity curves for BT474 and UACC893 cells are provided in FIG. 1B. Both Compound A and Compound B increased caspase 3/7 activity in BT474 and UACC893 cells as single agents. A slight increase in caspase 3/7 activity was observed with Compound A or Compound B as single agents in HCC1419 cells. The combination of Compound A and Compound B enhanced the levels of observed caspase 3/7 activity in BT474, UACC893 and HCC1419 cell lines.

TABLE 1
Cell growth inhibition by Compound A, Compound B and their combination in tumor cell lines.

| | | | PIK3CA | Single Agent (IC₅₀, μM) | |
|---|---|---|---|---|---|
| Tumor Type | Cell Line | HER2 Status | Status | Compound A | Compound B |
| Breast | HCC2218 | HER2+ | WT | 0.029 +/− 0.010 | 0.016 +/− 0.009 |
| Breast | HCC1419 | HER2+ | WT | 0.095 +/− 0.050 | 0.017 +/− 0.007 |
| Breast | BT-474 | HER2+ | K111N | 0.241 +/− 0.227 | 0.028 +/− 0.014 |
| Breast | SK-BR-3 | HER2+ | WT | 0.312 +/− 0.077 | 0.011 +/− 0.003 |
| Breast | KPL4 | HER2+ | H1047R | 0.433 +/− 0.053 | 0.003 +/− 0.000 |
| Breast | UACC893 | HER2+ | H1047R | 1.068 +/− 0.520 | 0.005 +/− 0.003 |
| Breast | MDA-MB-453 | HER2+ | H1047R | 2.467 +/− 0.145 | 0.015 +/− 0.002 |
| Breast | BT-474-J4* | HER2+ | K111N | 3.541 +/− 3.686 | 0.014 +/− 0.008 |
| Breast | HCC202 | HER2+ | E545K | 4.627 +/− 3.586 | 0.206 +/− 0.166 |
| Breast | SK-BR-3-W13* | HER2+ | WT | 7.109 +/− 4.374 | 0.167 +/− 0.098 |
| Breast | JIMT-1* | HER2+ | WT | 9.620 +/− 1.179 | 0.028 +/− 0.008 |
| Breast | MDA-MB-361* | HER2+ | E545K | >10 | 0.009 +/− 0.002 |
| Breast | MDA-MB-175-VII | HER− | WT | 0.106 +/− 0.008 | 0.009 +/− 0.002 |
| Breast | MDA-MB-415 | HER− | WT | 5.814 +/− 3.535 | 0.034 +/− 0.039 |
| Breast | HCC1569 | HER− | WT | 6.871 +/− 5.419 | 0.045 +/− 0.025 |
| Breast | HCC1937 | HER− | WT | 10.910 +/− 3.973 | 0.131 +/− 0.016 |
| Breast | HCC38 | HER− | WT | >10 | 0.097 +/− 0.016 |
| Breast | MDA-MB-157 | HER− | WT | >10 | 1.317 +/− 1.098 |
| Breast | HCC1806 | HER− | WT | >10 | 0.124 +/− 0.058 |
| Breast | ZR-75-1 | HER− | WT | >10 | 0.012 +/− 0.011 |
| Colon | NCI-H508 | HER− | E545K | 0.048 +/− 0.027 | 0.005 +/− 0.002 |
| Melanoma | CHL-1 | HER− | WT | 0.189 +/− 0.002 | 0.038 +/− 0.022 |
| Lung | CALU-3 | HER2+ | WT | 0.386 +/− 0.164 | 0.019 +/− 0.004 |

| | | Combination (Compound A:Compound B = 10:1) | | | |
|---|---|---|---|---|---|
| Tumor | | Compound A | Compound B | CI | |
| Type | Cell Line | (IC50, μM) | (IC50, μM) | Mut non Ex | EOHSA (ppts) |
| Breast | HCC2218 | 0.018 +/− 0.004 | 0.002 +/− 0.000 | 0.85 +/− 0.13 | 13.70 +/− 6.80 |
| Breast | HCC1419 | 0.032 +/− 0.014 | 0.003 +/− 0.001 | 0.64 +/− 0.21 | 15.80 +/− 5.39 |
| Breast | BT-474 | 0.029 +/− 0.014 | 0.003 +/− 0.001 | 0.34 +/− 0.22 | 15.44 +/− 5.71 |
| Breast | SK-BR-3 | 0.029 +/− 0.004 | 0.003 +/− 0.000 | 0.39 +/− 0.08 | 23.39 +/− 4.56 |
| Breast | KPL4 | 0.017 +/− 0.001 | 0.002 +/− 0.000 | 0.67 +/− 0.02 | 22.68 +/− 2.26 |
| Breast | UACC893 | 0.027 +/− 0.016 | 0.003 +/− 0.002 | 0.50 +/− 0.15 | 27.58 +/− 15.68 |
| Breast | MDA-MB-453 | 0.061 +/− 0.004 | 0.006 +/− 0.000 | 0.45 +/− 0.03 | 24.95 +/− 4.21 |
| Breast | BT-474-J4* | 0.040 +/− 0.027 | 0.004 +/− 0.003 | 0.31 +/− 0.08 | 20.37 +/− 4.06 |
| Breast | HCC202 | 0.323 +/− 0.427 | 0.032 +/− 0.043 | 0.23 +/− 0.14 | 22.12 +/− 6.95 |
| Breast | SK-BR- | 0.620 +/− 0.290 | 0.062 +/− 0.029 | 0.55 +/− 0.11 | 8.80 +/− 3.77 |
| | 3-W13* | | | | |
| Breast | JIMT-1* | 0.184 +/− 0.037 | 0.018 +/− 0.004 | 0.71 +/− 0.10 | 7.64 +/− 4.20 |
| Breast | MDA-MB- | 0.071 +/− 0.036 | 0.007 +/− 0.004 | N/A | 8.15 +/− 7.05 |
| | 361* | | | | |
| Breast | MDA-MB- | 0.025 +/− 0.006 | 0.003 +/− 0.001 | 0.59 +/− 0.18 | 33.16 +/− 10.65 |
| | 175-VII | | | | |
| Breast | MDA-MB-415 | 0.265 +/− 0.317 | 0.026 +/− 0.032 | 0.78 +/− 0.16 | 5.60 +/− 1.39 |
| Breast | HCC1569 | 0.230 +/− 0.190 | 0.023 +/− 0.019 | N/A | 17.91 +/− 9.11 |
| Breast | HCC1937 | 0.751 +/− 0.202 | 0.075 +/− 0.020 | 0.61 +/− 0.27 | 9.82 +/− 7.10 |
| Breast | HCC38 | 0.602 +/− 0.139 | 0.060 +/− 0.014 | N/A | 7.37 +/− 1.34 |
| Breast | MDA-MB-157 | 4.199 +/− 2.311 | 0.420 +/− 0.231 | N/A | 5.42 +/− 3.07 |
| Breast | HCC1806 | 0.782 +/− 0.364 | 0.078 +/− 0.036 | N/A | 9.77 +/− 2.32 |
| Breast | ZR-75-1 | 0.098 +/− 0.079 | 0.010 +/− 0.008 | N/A | 3.61 +/− 5.21 |
| Colon | NCI-H508 | 0.014 +/− 0.014 | 0.001 +/− 0.001 | 0.58 +/− 0.44 | 18.21 +/− 9.86 |
| Melanoma | CHL-1 | 0.056 +/− 0.012 | 0.006 +/− 0.001 | 0.51 +/− 0.01 | 27.70 +/− 0.41 |
| Lung | CALU-3 | 0.054 +/− 0.007 | 0.005 +/− 0.001 | 0.48 +/− 0.09 | 26.55 +/− 1.35 |

*lines resistant to Compound A and trastuzumab;
N/A: not applicable;
Combination index: CI.

### Example 1

### Capsule Composition

An oral dosage form for administering a combination of the present invention is produced by filing a standard two piece hard gelatin capsule with the ingredients in the proportions shown in Table I, below.

TABLE I

| INGREDIENTS | AMOUNTS |
|---|---|
| N-{3-Chloro-4-[(3-fluorobenzyl)oxy]phenyl}-6-[5-({[2- | 250 mg |
| (methanesulphonyl)ethyl]amino}methyl)-2-furyl]-4- | |
| quinazolinamine ditosylate monohydrate (the ditosylate | |
| monohydrate salt of Compound A) | |
| 4-difluoro-N-{2-(methyloxy)-5-[4-(4-pyridazinyl)-6- | 300 mg |
| quinolinyl]-3-pyridinyl} benzenesulfonamide | |
| Mannitol | 250 mg |
| Talc | 125 mg |
| Magnesium Stearate | 8 mg |

### Example 2

### Capsule Composition

An oral dosage form for administering one of the compounds of the present invention is produced by filing a standard two piece hard gelatin capsule with the ingredients in the proportions shown in Table II, below.

TABLE II

| INGREDIENTS | AMOUNTS |
|---|---|
| N-{3-Chloro-4-[(3-fluorobenzyl)oxy]phenyl}-6-[5-({[2- | 250 mg |
| (methanesulphonyl)ethyl]amino}methyl)-2-furyl]-4- | |
| quinazolinamine ditosylate monohydrate (the ditosylate | |
| monohydrate salt of Compound A) | |
| Mannitol | 55 mg |
| Talc | 16 mg |
| Magnesium Stearate | 4 mg |

### Example 3

### Capsule Composition

An oral dosage form for administering one of the compounds of the present invention is produced by filing a standard two piece hard gelatin capsule with the ingredients in the proportions shown in Table III, below.

TABLE III

| INGREDIENTS | AMOUNTS |
|---|---|
| 4-difluoro-N-{2-(methyloxy)-5-[4-(4-pyridazinyl)- | 300 mg |
| 6-quinolinyl]-3-pyridinyl} benzenesulfonamide | |
| Mannitol | 250 mg |
| Talc | 125 mg |
| Magnesium Stearate | 8 mg |

### Example 4

### Tablet Composition

The sucrose, microcrystalline cellulose and the compounds of the invented combination, as shown in Table IV below, are mixed and granulated in the proportions shown with a 10% gelatin solution. The wet granules are screened, dried, mixed with the starch, talc and stearic acid, then screened and compressed into a tablet.

TABLE IV

| INGREDIENTS | AMOUNTS |
|---|---|
| N-{3-Chloro-4-[(3-fluorobenzyl)oxy]phenyl}-6-[5-({[2- | 250 mg |
| (methanesulphonyl)ethyl]amino}methyl)-2-furyl]-4- | |
| quinazolinamine ditosylate monohydrate (the ditosylate | |
| monohydrate salt of Compound A) | |
| 4-difluoro-N-{2-(methyloxy)-5-[4-(4-pyridazinyl)-6- | 300 mg |
| quinolinyl]-3-pyridinyl} benzenesulfonamide | |
| Microcrystalline cellulose | 300 mg |
| sucrose | 10 mg |
| starch | 40 mg |
| talc | 20 mg |
| stearic acid | 5 mg |

### Example 5

### Tablet Composition

The sucrose, microcrystalline cellulose and one of the compounds of the invented combination, as shown in Table V below, are mixed and granulated in the proportions shown with a 10% gelatin solution. The wet granules are screened, dried, mixed with the starch, talc and stearic acid, then screened and compressed into a tablet.

TABLE V

| INGREDIENTS | AMOUNTS |
|---|---|
| N-{3-Chloro-4-[(3-fluorobenzyl)oxy]phenyl}-6-[5-({[2- | 250 mg |
| (methanesulphonyl)ethyl]amino}methyl)-2-furyl]-4- | |
| quinazolinamine ditosylate monohydrate (the ditosylate | |
| monohydrate salt of Compound A) | |
| Microcrystalline cellulose | 30 mg |
| sucrose | 4 mg |
| starch | 2 mg |
| talc | 1 mg |
| stearic acid | 0.5 mg |

### Example 6

### Tablet Composition

The sucrose, microcrystalline cellulose and one of the compounds of the invented combination, as shown in Table VI below, are mixed and granulated in the proportions shown with a 10% gelatin solution. The wet granules are screened, dried, mixed with the starch, talc and stearic acid, then screened and compressed into a tablet.

TABLE VI

| INGREDIENTS | AMOUNTS |
|---|---|
| 4-difluoro-N-{2-(methyloxy)-5-[4-(4-pyridazinyl)- | 300 mg |
| 6-quinolinyl]-3-pyridinyl} benzenesulfonamide | |
| Microcrystalline cellulose | 300 mg |
| sucrose | 40 mg |
| starch | 20 mg |
| talc | 10 mg |
| stearic acid | 5 mg |

While the preferred embodiments of the invention are illustrated by the above, it is to be understood that the invention is not limited to the precise instructions herein disclosed and that the right to all modifications coming within the scope of the following claims is reserved.

1. A combination comprising:
(i) a compound of Structure (I): 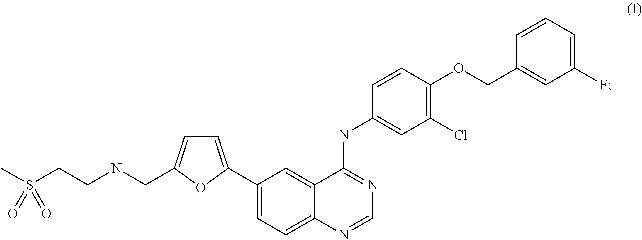 [SMILES: CS(=O)(=O)CCNCc1ccc(-c2ccc3ncnc(Nc4ccc(OCc5cccc(F)c5)c(Cl)c4)c3c2)o1]
or a pharmaceutically acceptable hydrate and/or salt thereof; and
(ii) a compound of Structure (II): 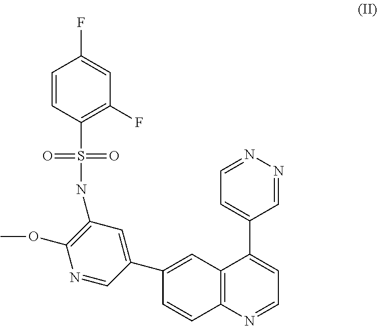 [SMILES: COc1ncc(-c2ccc3nccc(-c4ccnnc4)c3c2)cc1NS(=O)(=O)c1ccc(F)cc1F]
or a pharmaceutically acceptable salt thereof.

2. The combination according to claim 1 where the compound of Structure (I) is in the form of a ditosylate monohydrate salt.

3. A combination kit comprising the combination according to claim 1 together with a pharmaceutically acceptable carrier.

4. The combination according to claim 1 where the amount of the compound of Structure (I) is an amount selected from 250 mg to 1,500 mg, and that amount is administered once per day in one or more tablets, and the amount of the compound of Structure (II) is an amount selected from 0.25 mg to 75 mg, and that amount is administered once per day.

5. (canceled)

6. A method of treating cancer in a human in need thereof comprising administering to said human a therapeutically effective amount of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

7-9. (canceled)

10. The method of claim 6 wherein the cancer is selected from: gliomas, glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, gastrointestinal stromal tumorand testicular cancer.

11. The method according to claim 10 wherein the amount of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, is selected from about 250 mg to about 1,500 mg, and that amount is administered once per day in one or more tablets, and the amount of 4-difluoro-N-{2-(methyloxy)-5-[4-(4-pyridazinyl)-6-quinolinyl]-3-pyridinyl}benzenesulfonamide is selected from about 0.25 mg to about 75 mg, and that amount is administered once per day.

12. (canceled)

13. The method according to claim 11 wherein N-{3-Chloro-4-[(3-fluorobenzyl)oxy]phenyl}-6-[5-({[2-(methanesulphonyl)ethyl]amino}methyl)-2-furyl]-4-quinazolinamine ditosylate monohydrate and 4-difluoro-N-{2-(methyloxy)-5-[4-(4-pyridazinyl)-6-quinolinyl]-3-pyridinyl}benzenesulfonamide are administered within 12 hours of each other each day for a period of at least 7 consecutive days.

14. The method according to claim 10 wherein the cancer is selected from ovarian, breast, pancreatic and prostate.

15-17. (canceled)

18. A method of treating cancer in a human in need thereof wherein the cancer is either wild type or mutant for Raf and either wild type or mutant for PI3K/PTEN comprising administering to said human a therapeutically effective amount of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, and 4-difluoro-N-{2-(methyloxy)-5-[4-(4-pyridazinyl)-6-quinolinyl]-3-pyridinyl}benzenesulfonamide.

19. The method according to claim 18 wherein the amount of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, is selected from about 250 mg to about 1,500 mg, and that amount is administered once per day in one or more tablets, and the amount of 4-difluoro-N-{2-(methyloxy)-5-[4-(4-pyridazinyl)-6-quinolinyl]-3-pyridinyl}benzenesulfonamide is selected from about 0.25 mg to 75 mg, and that amount is administered once per day.

20. (canceled)

21. The method according to claim 19 wherein N-{3-Chloro-4-[(3-fluorobenzyl)oxy]phenyl}-6-[5-({[2-(methanesulphonyl)ethyl]amino}methyl)-2-furyl]-4-quinazolinamine ditosylate monohydrate and 2,4-difluoro-N-{2-(methyloxy)-5-[4-(4-pyridazinyl)-6-quinolinyl]-3-pyridinyl}benzenesulfonamide, or a pharmaceutically acceptable salt thereof are administered within 12 hours of each other each day for a period of at least 7 consecutive days.

22. The method according to claim 18 wherein the cancer is selected from ovarian, breast, pancreatic and prostate.

23-25. (canceled)

26. The method according to claim 10 wherein said N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof, and said 2,4-difluoro-N-{2-(methyloxy)-5-[4-(4-pyridazinyl)-6-quinolinyl]-3-pyridinyl}benzenesulfonamide, or a pharmaceutically acceptable salt thereof, are administered sequentially.

27. The method according to claim 26 wherein the amount of N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof thereof, is selected from about 250 mg to about 1,500 mg, and that amount is administered once per day in one or more tablets, and the amount of 4-difluoro-N-{2-(methyloxy)-5-[4-(4-pyridazinyl)-6-quinolinyl]-3-pyridinyl}benzenesulfonamide is selected from about 0.25 mg to 75 mg.

28. (canceled)

29. The method according to claim 27 wherein N-{3-Chloro-4-[(3-fluorobenzyl)oxy]phenyl}-6-[5-({[2-(methanesulphonyl)ethyl]amino}methyl)-2-furyl]-4-quinazolinamine ditosylate monohydrate is administered for from 1 to 30 consecutive days, followed by an optional drug holiday of from 1 to 14 days, followed by administration of 2,4-difluoro-N-{2-(methyloxy)-5-[4-(4-pyridazinyl)-6-quinolinyl]-3-pyridinyl}benzenesulfonamide, or a pharmaceutically acceptable salt thereof for from 1 to 30 days.

30. The method according to claim 26, wherein the cancer is selected from ovarian, breast, pancreatic and prostate.

31-45. (canceled)

46. The method according to claim 26, wherein 2,4-difluoro-N-{2-(methyloxy)-5-[4-(4-pyridazinyl)-6-quinolinyl]-3-pyridinyl}benzenesulfonamide, or a pharmaceutically acceptable salt thereof is administered for 1 or 2 days, and N-{3-Chloro-4-[(3-fluorobenzyl)oxy]phenyl}-6-[5-({[2-(methanesulphonyl)ethyl]amino}methyl)-2-furyl]-4-quinazolinamine, or a pharmaceutically acceptable hydrate and/or salt thereof is administered for 2-14 consecutive days.

47-49. (canceled)

50. The method according to claim 46, wherein the cancer is selected from ovarian, breast, pancreatic and prostate.

51-53. (canceled)

54. The method according to claim 46, wherein N-{3-Chloro-4-[(3-fluorobenzyl)oxy]phenyl}-6-[5-({[2-(methanesulphonyl)ethyl]amino}methyl)-2-furyl]-4-quinazolinamine is administered as the ditosylate monohydrate thereof.

55-59. (canceled)
